(19) United States
(12) Patent Application Publication
(10) Pub. No.: US 2014/0058245 A1
(43) Pub. Date: Feb. 27, 2014

(54) MEASURING APPARATUS

(71) Applicants: Takuji Oishi, Kyoto-shi (JP); Kazuhiko Fukutani, Kyoto-shi (JP)

(72) Inventors: Takuji Oishi, Kyoto-shi (JP); Kazuhiko Fukutani, Kyoto-shi (JP)

(73) Assignee: CANON KABUSHIKI KAISHA, Tokyo (JP)

(21) Appl. No.: 14/009,020

(22) PCT Filed: Mar. 29, 2012
(86) PCT No.: PCT/JP2012/059299

§ 371 Date: Sep. 30, 2013

(30) Foreign Application Priority Data
Apr. 8, 2011 (JP) 2011-086569

(51) Int. Cl. A61B 5/00

(52) U.S. Cl. CPC A61B 5/0095
USPC 600/407

(57) ABSTRACT

A measuring apparatus is provided including a holding unit holding an object, an acoustic detecting unit including at least one detector which receives, via the holding unit, an acoustic wave generated from the object to which light is irradiated and converts the acoustic wave into an electrical signal, and a processor which generates image data of the object by using the electrical signal based on the acoustic wave received by the acoustic detecting unit at first and second measurement locations, wherein the acoustic detecting unit is arranged so as to form an overlapped area that is thicker than the object in a normal direction of an interface between the holding unit and the object as a result of the effective receiving areas of the detector in the first and second measurement locations overlapping in the object.

### TECHNICAL FIELD

The present invention relates to a measuring apparatus.

### BACKGROUND ART

An imaging apparatus which utilizes X-ray and ultrasound echo is being used in numerous fields that require nondestructive testing, a prominent example being the medical field. With an imaging apparatus used in the medical field, since physiological information, or functional information, of a living body is effective for discovering the diseased site of cancer or the like, research on the imaging of functional information is being conducted in recent years. As one diagnostic approach using functional information, photoacoustic tomography (PAT) as one type of optical imaging technology has been proposed. While only morphological information of the living body can be obtained with X-ray diagnosis or diagnosis using ultrasound echo, with photoacoustic tomography it is possible to obtain functional information in a non-invasive manner.

Photoacoustic tomography is technology which irradiates pulsed light generated from a light source to an object, and performs imaging of the acoustic wave generated from the body tissue that absorbed the optical energy which was propagated and dispersed within the object. In other words, the temporal change of the received acoustic wave is detected at a plurality of locations surrounding the object, and, by subjecting the obtained signals to mathematical analysis; that is, back projection, information relating to the optical characteristics in the object is visualized three-dimensionally.

Back projection is a calculation method of specifying the signal source by giving consideration to the propagation velocity of the acoustic wave in the object, propagating the respective received signals in reverse, and superimposing the signals. Based on this technology, it is possible to obtain an optical characteristic distribution such as the light absorption coefficient distribution of the living body from the initial pressure generation distribution in the object, and thereby obtain the internal information of the object. In particular, since near-infrared light can easily permeate water which configures most of the living body, and possesses properties of being easily absorbed by the hemoglobin in the blood, it can create an image of the blood vessels.

With photoacoustic tomography, there are those referred to as a planar type and a circular type depending on the positioning of the acoustic detectors. In other words, those in which the acoustic detectors are positioned on one planar surface are a planar type (Non Patent Literature 1: NPL 1), and those in which the acoustic detectors are positioned in a circle to surround the object are a circular type (Patent Literature 1: PTL 1). Both of these types have their respective characteristics, but a planar type allows the downsizing of the apparatus in cases of measuring something large like a human body.

### CITATION LIST

### Patent Literature

PTL 1: U.S. Patent Application Publication No. 2007/0238958

### Non Patent Literature

NPL 1: Srirang Manohar, et al. “Region-of-interest breast studies using the Twente Photoacoustic Mammoscope (PAM)” Proc. of SPIE Vol. 6437 (2007) 643702-9

### SUMMARY OF INVENTION

### Technical Problem

The planar type and the circular type respectively have the following problems in terms of resolution.

When performing back projection by using the propagation velocity of the acoustic wave in the object, with a planar type such as NPL 1, the lateral resolution and the sensitivity will be a trade-off relationship. With a planar type, the resolution (lateral resolution) of the direction that is parallel to the acoustic detector face is mainly decided by the width of the elements of the acoustic detector, and the resolution (depth resolution) of the direction that is perpendicular to the acoustic detector face is decided by the frequency of the elements. If the width of the elements is reduced in order to improve the lateral resolution, the receiving surface area of the acoustic wave will decrease, and the sensitivity will deteriorate. Thus, the lateral resolution and the sensitivity are of a trade-off relationship. Since there is a limit in improving the lateral resolution as described above, generally speaking the depth resolution has a higher resolution than the lateral resolution.

Meanwhile, a circular type such as PTL 1 has a higher resolution than the planar type since it can receive signals from the object at all angles, but the resolution is subject to location dependency, and the resolution becomes inferior as it goes outward from the center of the circle. Since the front face of an acoustic detector has strong receiving sensitivity, and since all acoustic detectors are facing the center of the circle with a circular type, an acoustic wave that is generated near the center is detected by all acoustic detectors. Upon superimposing the received signals of the respective detectors based on back projection, the detectors are arranged to surround the periphery, and the information of the depth direction of all detectors will be superimposed. Thus, the lateral resolution and the depth resolution will be equal. Meanwhile, at the outside away from the center of the circle, only certain acoustic detectors will have sensitivity, and only the received signals of certain detectors can be used in the back projection. In addition, since the angles of these detectors are close, the result is similar to a planar type. Accordingly, the lateral resolution approaches the lateral resolution of a planar type as it nears the outside of the circle, and the resolution will deteriorate in comparison to the vicinity of the center.

The present invention was devised in view of the foregoing problems, and its object is to provide a measuring apparatus capable of obtaining high resolution while maintaining sensitivity without any location dependency.

### Solution to Problem

In order to achieve the foregoing object, the present invention provides a measuring apparatus, comprising:

a holding unit holding an object;

an acoustic detecting unit including at least one detector which receives, via the holding unit, an acoustic wave that is generated from the object to which light is irradiated and converts the acoustic wave into an electrical signal; and

a processor generating image data of the object by using the electrical signal based on the acoustic wave that has been received by the acoustic detecting unit at a first measurement location and a second measurement location,

wherein the acoustic detecting unit is arranged so as to form an overlapped area in which an effective receiving area of the detector in the first measurement location and an effective receiving area of the detector in the second measurement location overlap in the object.

### Advantageous Effects of Invention

According to the present invention, it is possible to provide a measuring apparatus capable of obtaining high resolution while maintaining sensitivity without any location dependency.

Further features of the present invention will become apparent from the following description of exemplary embodiments with reference to the attached drawings.

### BRIEF DESCRIPTION OF DRAWINGS

FIG. 1 is a block diagram showing the configuration of an embodiment of the present invention.

FIG. 2 is a flowchart showing a method of implementing an embodiment of the present invention.

FIG. 3 is a diagram showing the arrangement of an embodiment of the present invention.

FIG. 4A is a diagram showing the arrangement of an embodiment of the present invention.

FIG. 4B is a diagram showing the arrangement of an embodiment of the present invention.

FIG. 5 is a diagram showing the definitions that are used for explaining the arrangement of the present invention.

FIG. 6 is a diagram showing the arrangement of an embodiment of the present invention.

FIG. 7 is a diagram showing the arrangement of an embodiment of the present invention.

FIG. 8A is a diagram showing the arrangement of an embodiment of the present invention.

FIG. 8B is a diagram showing the arrangement of an embodiment of the present invention.

FIG. 9 is a flowchart showing a method of implementing an embodiment of the present invention.

FIG. 10 is a diagram showing the arrangement of an embodiment of the present invention.

FIG. 11 is a flowchart showing a method of implementing an embodiment of the present invention.

FIG. 12 is a diagram showing the arrangement of an embodiment of the present invention.

FIGS. 13A and 13B are diagrams showing the calculation results of the sound pressure distribution for explaining the Examples.

### DESCRIPTION OF EMBODIMENTS

### Embodiment 1

The basic embodiments of the present invention are now explained with reference to the drawings. In the ensuing embodiments, an imaging apparatus employing the photoacoustic tomography technology is explained as the measuring apparatus.

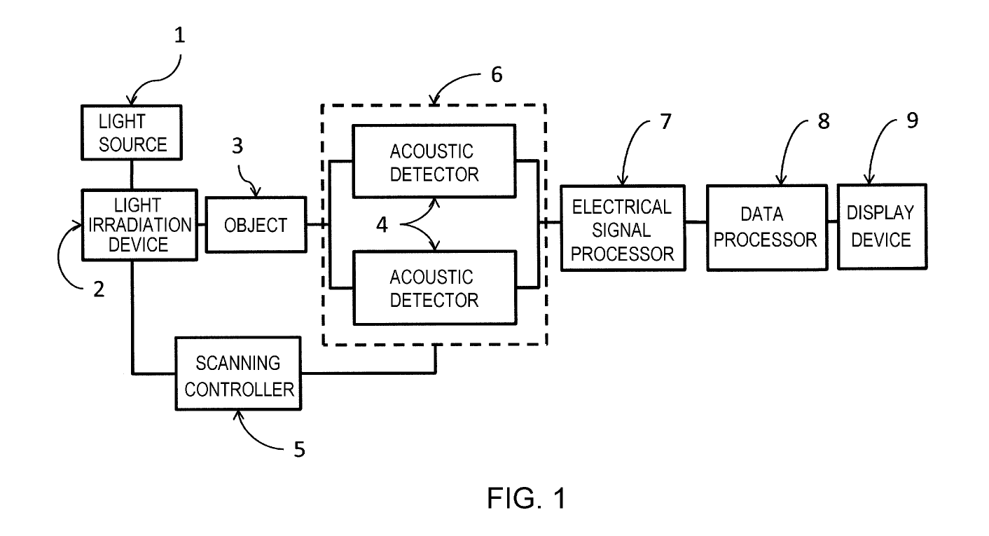

FIG. 1 shows the first embodiment of the imaging apparatus of the present invention. The target to be measured by the imaging apparatus is an object 3.

The imaging apparatus in this embodiment includes a light source 1 which generates pulsed light, a light irradiation device 2 which guides the pulsed light generated by the light source 1 to the object 3, and a plurality of acoustic detectors 4 which convert the acoustic wave that was excited by the pulsed light into an electrical signal. The imaging apparatus additionally includes a scanning controller 5 which associates and moves the light irradiation device 2 and the plurality of acoustic detectors 4, and an electrical signal processor 7 which amplifies the electrical signal from the acoustic detector, and A/D-converts and stores the electrical signal. The imaging apparatus is further configured from a data processor 8 which performs back projection using digital signals and thereby generates image data relating to the internal information of the object, and a display device 9 for displaying the results.

Note that with the acoustic detectors 4, a plurality of elements for detecting the acoustic wave are arranged in the in-plane direction, and signals from a plurality of locations can be obtained at once. Moreover, the plurality of acoustic detectors 4 configure a detection unit 6, and the relative positions of the plurality of acoustic detectors 4 are fixed. In the case of this embodiment, the acoustic detecting unit is configured from the plurality of acoustic detectors 4.

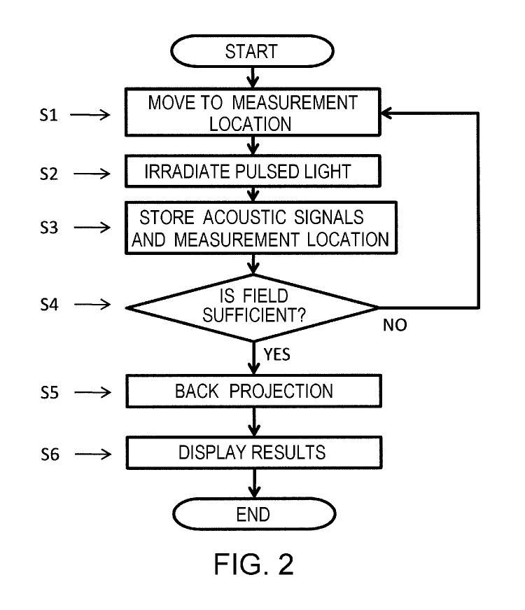

The implementation method is now explained with reference to FIG. 1 and FIG. 2.

FIG. 2 is a flowchart showing the method of implementing the present invention.

Foremost, the scanning controller 5 moves the light irradiation device 2 and the acoustic detector 4 so that the measurement target area of the object 3 can be measured (step S1). The acoustic detector 4 is moved together with the detection unit 6 so that the relative placement of the respective acoustic detectors is not changed as described later. Here, desirably the light irradiation device 2 is also synchronized and caused to perform scanning.

Subsequently, pulsed light is irradiated from the light irradiation device 2 (step S2). An acoustic wave generated from the object based on a photo-acoustic effect is received by a plurality of acoustic detectors 4 (planar array-type acoustic detectors) and converted into an electrical signal. The electrical signal is amplified by the electrical signal processor 7, subject to A/D conversion, and digital data is used as the acoustic signal.

Subsequently, the digital data is stored in a memory or the like (step S3). Here, the measured position is simultaneously stored. Note that the area that can be measured at once will depend on the size of the planar array-type acoustic detectors 4 and the installation method described later.

Subsequently, whether the measuring area that was measured in the object 3 has reached the intended range is determined (step S4). If the measuring area has not reached the intended range (S4=NO), S1 to S3 are repeated until the measured area reaches the intended range.

If the measuring area has reached the intended size (S4=YES), back projection is performed based on the stored digital data and information of the respective measurement locations, and sound pressure distribution (initial sound pressure distribution) upon the generation of the acoustic wave is created (step S5). Here, the internal distribution of the object that is created as image data in the present invention is not limited to the initial sound pressure distribution in the object, and may also be the light energy absorption density distribution that is derived from the initial sound pressure distribution, the absorption coefficient distribution, or the concentration distribution of the substance configuring the tissue. The concentration distribution of a substance is, for instance, oxygen saturation distribution or oxygenated and deoxygenated hemoglobin concentration distribution.

Finally, this distribution is displayed on the display device 9 (step S6).

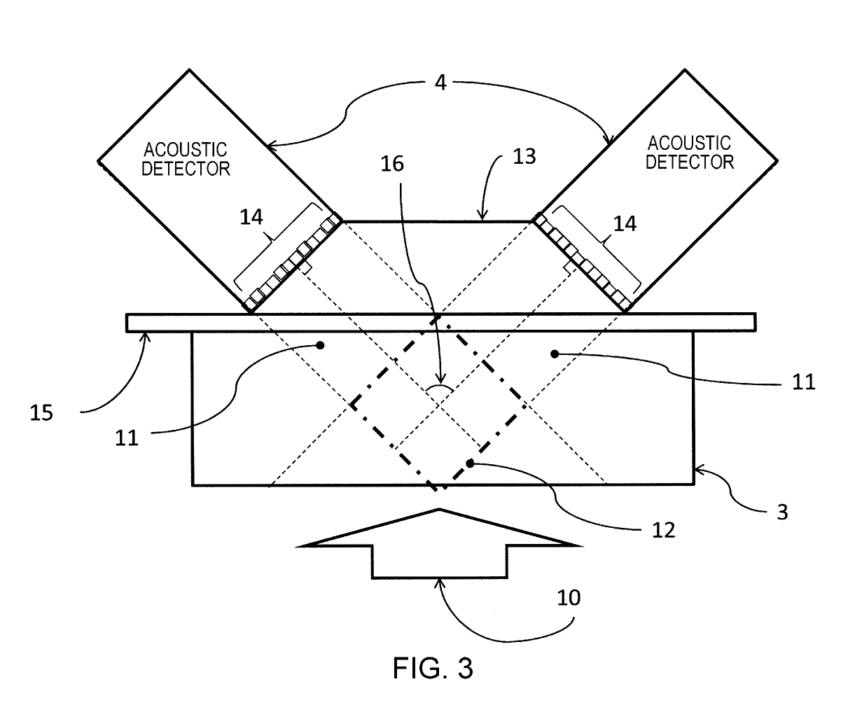

The installation method of the planar array-type acoustic detector according to the present invention is now explained with reference to FIG. 3 to FIG. 7.

FIG. 3 is a diagram showing the arrangement of the acoustic detectors and the object. The acoustic detector is a planar array-type acoustic detector in which a plurality of elements 14 are arranged on one planar surface, and the receiving surface thereof; that is, the face where the elements are arranged is in contact with an object holding plate 15 via an acoustic wave propagation medium 13. The object holding plate 15 is a holding unit for holding the object 3. The two acoustic detectors 4 can be respectively referred to as a first detector that is arranged at a first measurement location, and a second detector that is arranged at a second measurement location.

The acoustic wave propagation medium 13 and the object holding plate 15 desirably match the acoustic impedance of the object 3 and the acoustic detector 4, and are transparent relative to the light 10. When the object 3 is a living body, water can be used as the acoustic wave propagation medium 13, and a resin material can be used as the object holding plate 15.

The light 10 irradiated from the light irradiation device 2 is desirably irradiated from a region that is close to the measuring area. Here, light is irradiated from the opposite side of the acoustic detector across from the object so that the acoustic wave generated at the object interface does not overlap with the acoustic wave generated inside the object. However, if sufficient light will reach the measuring area, the light 10 may be irradiated from any region. As the light irradiation device 2 of the present invention, for example, used may be a mirror which reflects light, a lens which focuses, magnifies and changes the shape of light, a prism which disperses, refracts and reflects light, an optical fiber which propagates light, a diffuser panel, or the like. When the light source is compact such as a semiconductor laser, the light source itself may be used as the light irradiation device so as to directly irradiate light from the light source to the object.

The acoustic detectors 4 have directionality, and the sensitivity will deteriorate as the angle increases from the front face direction (direction that is perpendicular to the receiving surface). Here, the effective receiving area of the acoustic detector 4 is defined as an area within the angle where the sensitivity is 50 percent relative to the maximum receiving sensitivity of the front face of the acoustic detector. The directionality is decided based on the center frequency and size of the acoustic detector. In the diagram, the effective receiving area 11 is shown as the area within the range of the dotted lines which extend perpendicularly from both ends of the receiving surface on which the plurality of elements 14 are arranged. However, depending on the measurement, there are cases where sufficient sensitivity can be obtained even if the sensitivity is less than 50 percent. In the foregoing case, the effective receiving area shall be an area with sufficient sensitivity for performing the measurement.

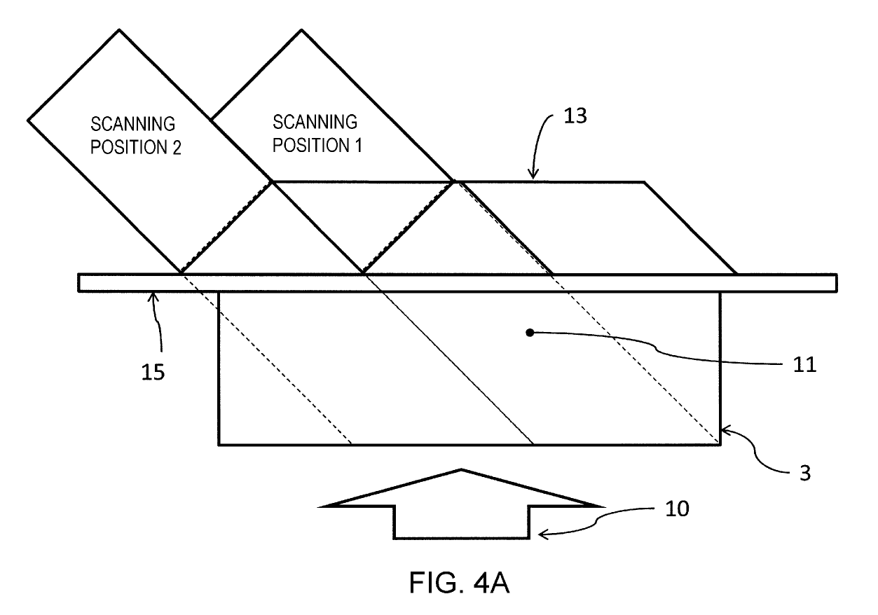

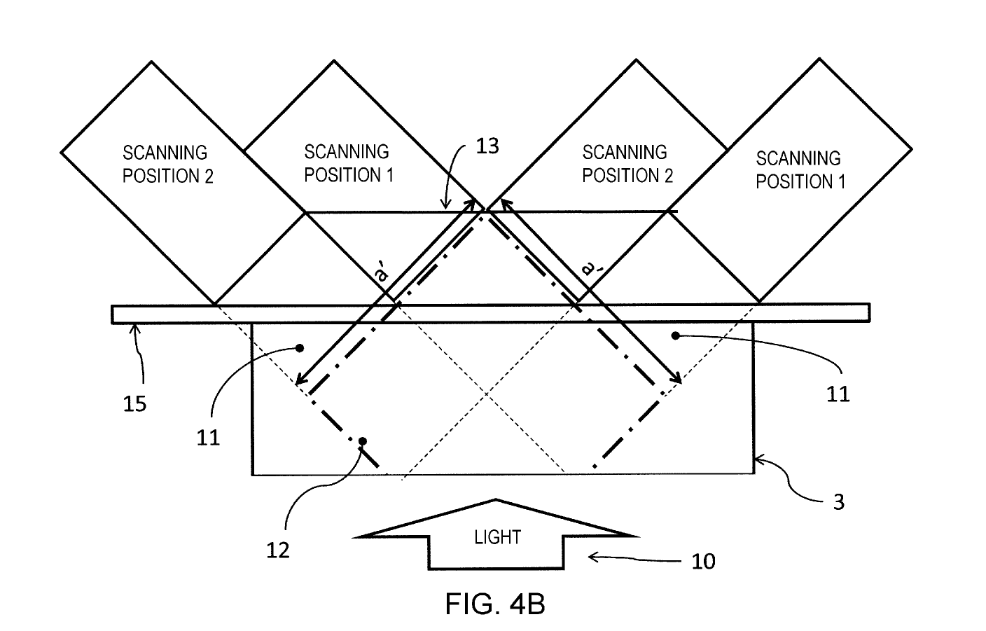

When the acoustic detector 4 is caused to perform scanning, the total area of the effective receiving areas 11 in the respective scanning positions (scanning position 1, scanning position 2) as shown in FIG. 4A becomes the effective receiving area of that acoustic detector. Two acoustic detectors 4 are provided as shown in FIG. 3, and are installed so that their effective receiving areas 11 overlap within the object 3. The range that is measured by all acoustic detectors; that is, the area that is formed as a result of the effective receiving areas of all acoustic detectors overlapping is defined as the overlapped area.

In FIG. 3, the overlapped area 12 is the portion that is surrounded by a thick dashed line within the effective receiving area 11. In addition, in order to eliminate the location dependency of the resolution, the overlapped area is formed to have a depth that is greater than the depth of the object (vertical direction in FIG. 3). The angle, size and scanning width of the acoustic detector, distance of the acoustic detector from the object, and distance between the acoustic detectors are adjusted so that the overlapped area will have the foregoing depth. Here, the acoustic detectors need to be installed at mutually crossing angles.

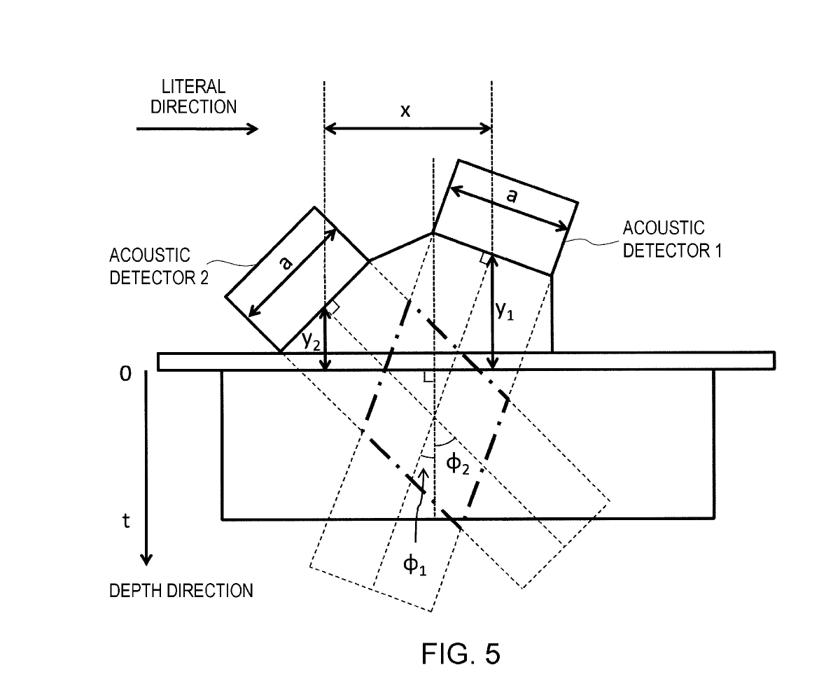

This is now represented as formulae with reference to FIG. 5. As shown in FIG. 5, with the interface of the object and the object holding plate as the zero point of the depth direction, the object thickness is t, the angle of the acoustic detector 1 relative to the normal of the interface of the object and the object holding plate is φ1, and similarly the angle of the acoustic detector 2 is φ2. Moreover, the distance of the depth direction of the center of the receiving surface of the acoustic detector 1 from the interface of the object and the object holding plate is y1, and similarly the distance of the depth direction of the center of the receiving surface of the acoustic detector 1 is y2. Moreover, the lateral direction distance of the center of the receiving surface of the acoustic detector 1 and the acoustic detector 2 is x, and the width of the acoustic detector 1 and the acoustic detector 2 is a. Here, the acoustic detectors 1, 2 are installed so as to satisfy following Formula (1) and Formula (2).

$$\begin{matrix}[ Math . 1 ] & \\ \frac{x - {y}_{1} tan {ϕ}_{1} - {y}_{2} tan {ϕ}_{2}}{tan {ϕ}_{1} + tan {ϕ}_{2}} - \frac{a cos ( \frac{{ϕ}_{1} + {ϕ}_{2}}{2} )}{2 sin ( \frac{{ϕ}_{1} - {ϕ}_{2}}{2} )} \leq 0 & ( 1 ) \\ t \leq \frac{x - {y}_{1} tan {ϕ}_{1} - {y}_{2} tan {ϕ}_{2}}{tan {ϕ}_{1} + tan {ϕ}_{2}} + \frac{a cos ( \frac{{ϕ}_{1} + {ϕ}_{2}}{2} )}{2 sin ( \frac{{ϕ}_{1} - {ϕ}_{2}}{2} )} & ( 2 )\end{matrix}$$

When (the detection unit of) the acoustic detector is caused to perform scanning, the overlapped area will be as shown in FIG. 4B. Here, considering that the width a of the acoustic detector is now a′ due to the scanning, the acoustic detectors 1, 2 are installed to satisfy Formula (1) and Formula (2).

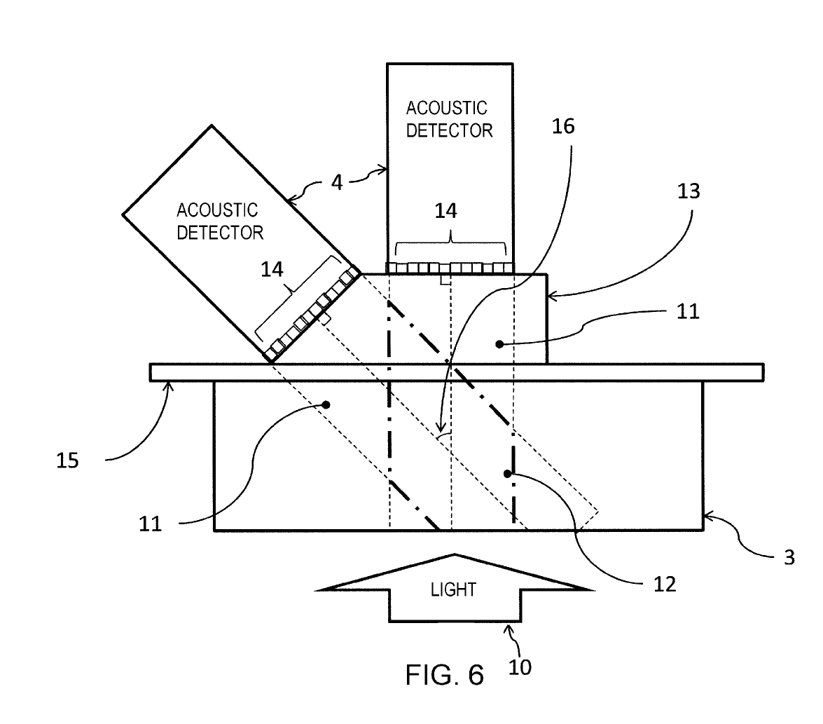

The acoustic detector 4 is desirably installed such that the central axis of the effective receiving area is line-symmetric relative to the normal of the object holding plate 15, but it may also be asymmetrical as shown FIG. 6. If the acoustic detector is a two-dimensional array with the elements arranged on a planar surface, the normal direction of the planar surface basically becomes the central axis of the effective receiving area.

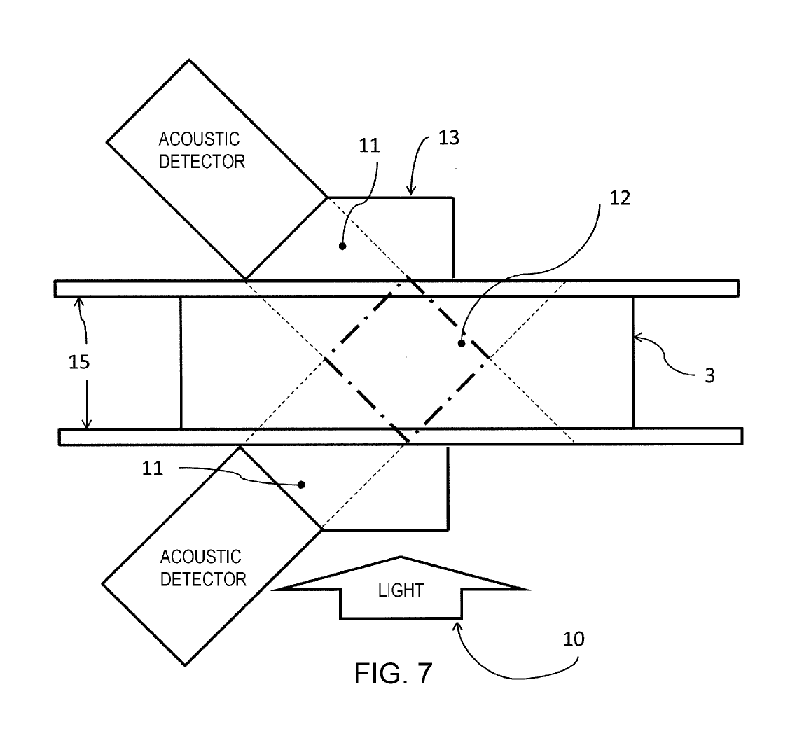

In addition, as shown in FIG. 7, it is also possible to provide two members as the object holding plate 15 on either side of the object, and provide acoustic detectors 4 of different angles on either side. Aiming to improve the acoustic consistency, it is also possible to interpose an acoustic matching material such as gel between the object holding plate and the object.

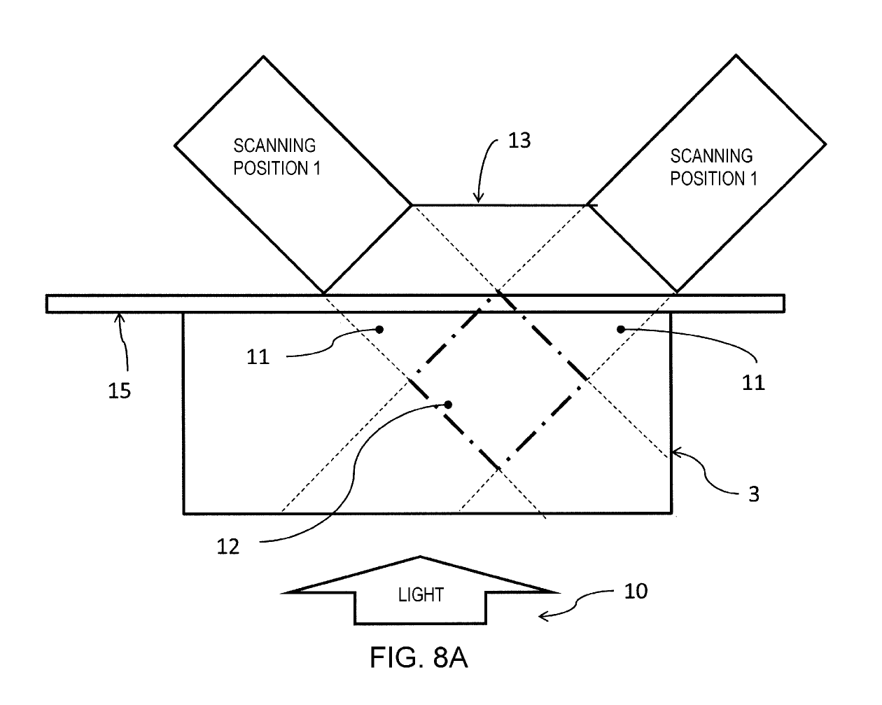

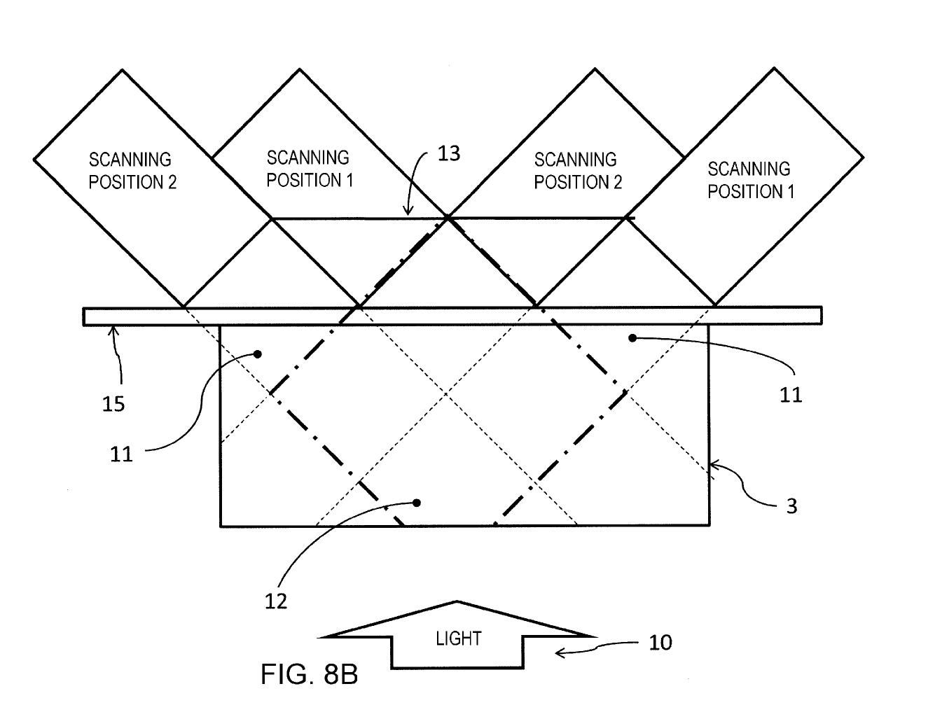

Moreover, as shown in FIG. 8A, even in cases where the overlapped area falls short of the thickness of the object, the size of the overlapped area can be enlarged to be greater than the thickness of the object and thereby measured by causing the acoustic detector to perform scanning as shown in FIG. 8B.

When the crossing angle of the acoustic detector; that is, when φ1-φ2 is 90 degrees and the signals of the respective elements are subject to back projection from the position of the respective elements, the lateral resolution and the depth resolution will become equal since the overlapped area 12 will be viewed as the mutual depth resolutions of the acoustic detectors. When comparing this with a planar type having the same element size, it is possible to realize high resolution while maintaining sensitivity. In addition, since the elements of the acoustic detector are arranged on a planar surface, the depth resolution in the effective receiving area 11, which is the front face of the elements, will be uniform without any location dependency, and this will also be uniform in the overlapped area 12 since depth resolutions that are free from location dependency are overlapped.

Moreover, with photoacoustic tomography, since the advancing direction of the sound wave will differ depending on the shape of the light absorber, there are cases where it is not possible to reproduce the shape of the light absorber only with acoustic detectors that are arranged in one direction. Nevertheless, since a plurality of acoustic detectors are facing mutually different directions in the present invention, a secondary effect of being able to complementarily reproducing the shape of the light absorber is yielded.

In addition, there are cases where the distribution obtained with a planar type back projection shows a virtual image referred to as an artifact or a ghost due to lack of information. Nevertheless, the present invention is also able to reduce such virtual image by obtaining information from a plurality of directions.

### Embodiment 2

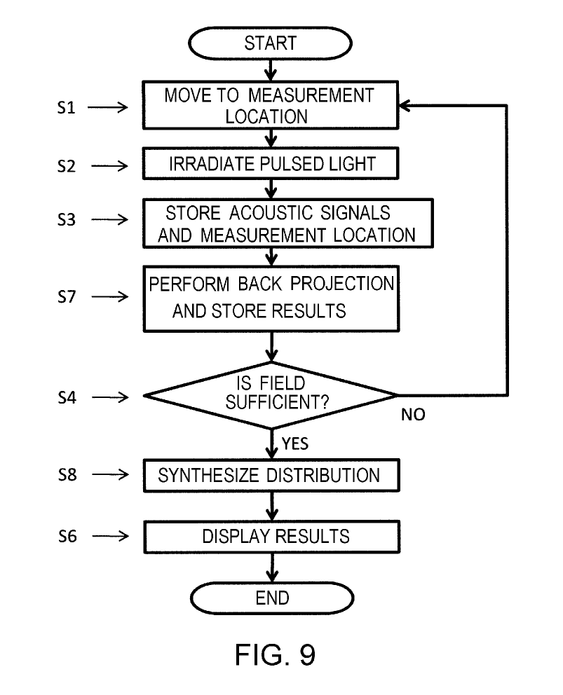

In Embodiment 2, the method of easily obtaining the initial sound pressure of the overlapped area is explained. The configuration and arrangement of the apparatus in this embodiment are the same as Embodiment 1, and only the method is different. The main differences with Embodiment 1 are now explained with reference to the flowchart of FIG. 9.

In steps S1 to S3, as with Embodiment 1, performed are scanning, irradiation of light, and storage of acoustic signals and positions.

Subsequently, the data processor 8 performs back projection using the signals and position of one of the acoustic detectors, obtains the initial sound pressure distribution of the effective receiving area, and stores the results (first image data). The data processor 8 thereafter similarly obtains the initial sound pressure distribution of the effective receiving area of the other acoustic detector, and stores the results (step S7, second image data).

Subsequently, whether the initial sound pressure distribution obtained from the respective acoustic detectors has reached the intended range is determined (step S4). If the intended range has not been reached (S4=NO), S1 to S3 and S7 are repeated until the intended range is reached.

If the intended range has been reached (S4=YES), the stored initial sound pressure distribution is synthesized (step S8). Since the initial sound pressure distribution is created for each acoustic detector, composition processing is performed upon creating the overlapped area. For the composition processing of the respective initial sound pressure distributions, preferably employed is a method of acquiring the square root of the product in which the overlapping effect is emphasized when the values are similar, but methods of acquiring the average or root-mean-square can also be adopted. It is thereby possible to generate image data of the object.

Finally, the results are displayed on the display device 9 (step S6).

In this embodiment, in order to simplify the back projection, the calculation time and resources of the computing device can be reduced.

### Embodiment 3

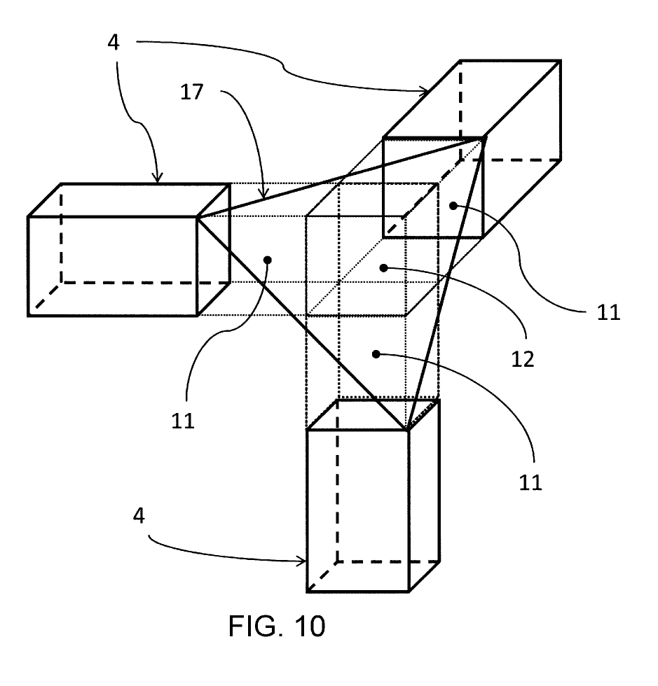

An example where Embodiment 1 is expanded three-dimensionally is now explained with reference to FIG. 10.

The configuration of the apparatus and the measurement method are the same as Embodiment 1 or Embodiment 2, and only the arrangement is different. Thus, the arrangement is now explained.

FIG. 10 is a diagram showing the arrangement of the acoustic detectors 4 in this embodiment. The planar surface 17 represents the object holding plate interface, and the near side of the plane of paper is the area where the object holding plate and the object exist. Here, for the convenience of viewing the drawings, although the planar surface 17 is only drawn in a range of connecting the corners of the acoustic detectors 4, it is also possible to expand the range on the same planar surface. The acoustic detector 4 is a planar array-type acoustic detector in which a plurality of elements are disposed in the same planar surface, and its receiving surface is in contact with the object holding plate interface 17 through an acoustic wave propagation medium not shown.

Light that is carried by the light irradiation device (not shown) is irradiated so that an amount sufficient for measurement reaches the measuring area. Three acoustic detectors 4 are provided, and each acoustic detector 4 has an effective receiving area 11 shown as a rectangle that is framed in by dotted lines. In addition, the acoustic detectors 4 are installed so that the effective receiving areas 11 overlap inside the object. The area where the effective receiving areas 11 respectively corresponding to the three acoustic detectors 4 overlap is the overlapped area 12. In addition, the acoustic detectors are installed to intersect with each other, and, desirably, they mutually form a crossing angle of 90 degrees. When the signals of the respective elements are subject to back projection from the position of the respective elements at a crossing angle of 90 degrees, it is possible to realize high resolution without any location dependency while maintaining planar type sensitivity in the overlapped area 12.

In this embodiment, high resolution is realized without any location dependency in all three-dimensional directions.

### Embodiment 4

The method of using only one acoustic detector among the two acoustic detectors used in Embodiment 1 is now explained.

The configuration of the apparatus of this embodiment is achieved by removing one of the two acoustic detectors used in Embodiment 1. Moreover, the arrangement of the two acoustic detectors in Embodiment 1 is referred to as measurement location 1 and measurement location 2, respectively. For example, upon removing one of the two acoustic detectors 4 in FIG. 3, if the remaining acoustic detector is on the left side, this is referred to as the measurement location 1 (first measurement location), and if it is on the right side, this is referred to as the measurement location 2 (second measurement location). In the case of this embodiment, the acoustic detecting unit is configured from one acoustic detector 4.

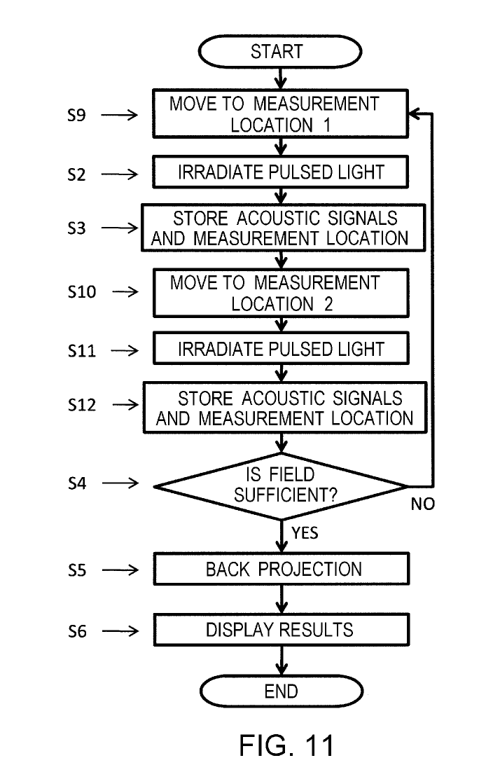

The implementation method is now explained with reference to the flowchart of FIG. 11.

In this embodiment, the acoustic detector is foremost moved to the measurement location 1 (step S9).

Subsequently, pulsed light is irradiated (step S2), and an acoustic signal is received and stored together with the measurement location (step S3).

The acoustic detector is thereafter moved to the measurement location 2 (step S10).

Then pulsed light is similarly irradiated (step S11), and an acoustic signal is received and stored together with the measurement location (step S12). The movement of the acoustic detector in the foregoing case is desirably carried out mechanically, but it may also be moved manually.

Subsequently, whether the measuring area has reached the intended range is determined (step S4).

If the measuring area has not reached the intended range (S4=NO), the measurement location 1 and the measurement location 2 are set so that different areas of the object can be measured, and S9, S2, S3, S10, S11, and S12 are repeated until the measuring area becomes the intended size.

When the measuring area reaches the intended size (S4=YES), back projection is performed using the stored signals and information on the measurement locations (step S5), and the results are displayed (step S6).

In this embodiment, it is possible to implement the present invention with one acoustic detector, and thereby reduce costs.

### Embodiment 5

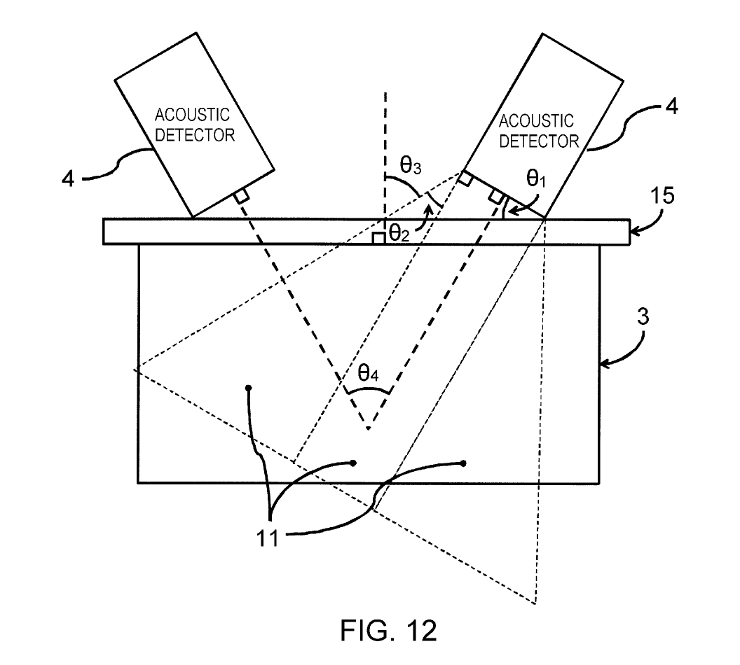

In this embodiment, setting a detector angle is now explained. As shown in FIG. 12, generally, effective receiving area 11 of the acoustic detector 4 is spread outside, not only in front of the acoustic detector 4, because of the directionality of the acoustic detector 4. The detector angle is set so that the effective receiving area 11 containing the spread outside area does not include total reflection area. Consequently, it is desirable that Formula (3) is satisfied, as shown in FIG. 14, when an angle between detection face of the acoustic detector 4 and the object holding plate 15 is defined as θ₁, the directional angle of the acoustic detector 4 is defined as θ₂, a critical angle of acoustic wave which is generated inside of the object 3 at a boundary between the object 3 and the object holding plate 15 is defined as θ₃, a crossing angle of the acoustic detector 4 is defined as θ₄.

0<θ₁≦θ₃−θ₂ (3)

Moreover, it is desirable that the detector angle θ₁ is set so that Formula (4) is satisfied because the resolution is higher when the crossing angle is more close to 90 degree.

θ₁=θ₃−θ₂ (4)

Moreover, it is desirable that the acoustic detector 4 is set in a line-symmetric relative to the normal of the object holding plate 15. In this case, Formula (5) is provided.

θ₄=2θ₁ (5)

Accordingly, the angle of the acoustic detector 4 is can be expressed as Formula (6).

θ₄=2θ₁=2(θ₃−θ₂) (6)

### EXAMPLES

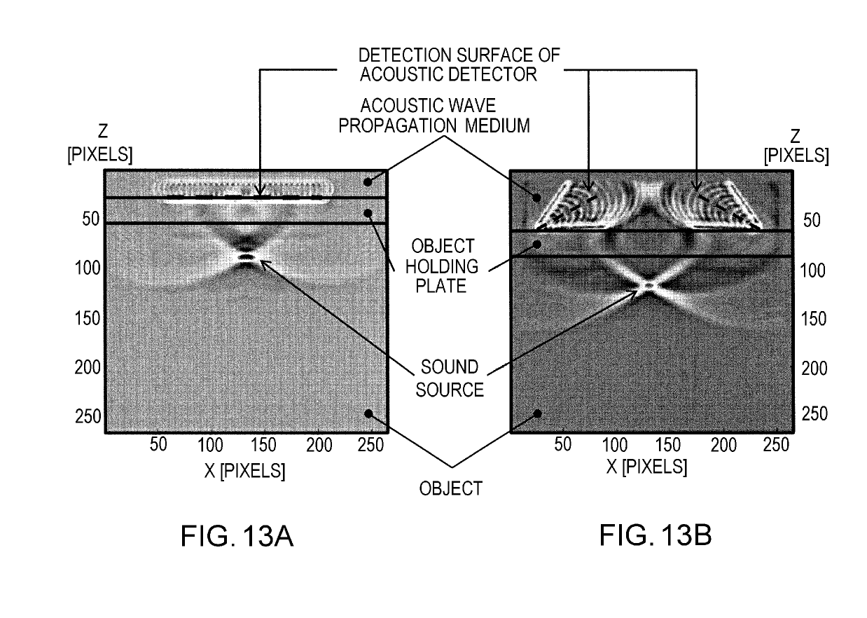

The results of implementing the present invention are shown using a two-dimensional simulation. Foremost, as a comparative example, the results of implementing a uniplanar type acoustic detector are shown, and the implementation results of the present invention are subsequently shown. Here, signals from a circular sound source to the detector position were simulated, and back projection using such signals was additionally performed to obtain the results. FIG. 13 is a diagram where the simulation system is overlapped on the results obtained from the back projection.

The planar type of the comparative example is now explained with reference to FIG. 13A. The acoustic detector is uniplanar, and has a width of 60 mm as a result of arranging 30 elements having a width of 2 mm. An object holding plate having a thickness of 10 mm was placed between the acoustic detector and the object in parallel to the acoustic detector, and the side that is farther from the acoustic detector was used as the object. The sound source is a circle having a diameter of 1 mm, and was placed at a location that is 20 mm away from the center when viewed from the acoustic detector; that is, a location that is 10 mm away from the interface of the object holding plate and the object. The propagation velocity of the sound wave was 2200 (m/s) in the object holding plate and 1500 (m/s) in the object, and the density was 0.83 (g/cm³) for the object holding plate and 1 (g/cm³) for the object.

Simulation was performed based on the foregoing system, and the obtained sound pressure distribution is shown in FIG. 13A. An image caused by acoustic interference appears in the acoustic wave propagation medium, but in reality attention is given only to the object, and only the inside of the object is obtained as the result. The dark portion shown at the center of the object is the sound source that is obtained based on the back projection.

Next, an example of implementing the present invention is explained with reference to FIG. 13B. Two acoustic detectors having a width of 30 mm as a result of arranging 15 elements having a width of 2 mm were prepared, and installed so that their mutual central parts are 57 mm apart and the crossing angle θ1-θ2 will be 60 degrees. Subsequently, as with the comparative example, an object holding plate having a thickness of 10 mm was placed, and the farther side was used as the object. The object holding plate was placed so that the crossing angle of the normal of the object holding plate and the normal of the acoustic detector receiving surface; that is, θ1, θ2 will be θ1=30 degrees, φ2=−30 degrees.

When only giving consideration to the resolution, the crossing angle of the acoustic detectors is desirably 90 degrees. Nevertheless, the absolute value of φ1, φ2 at such time will be 45 degrees, and the sound wave from the sound source will be totally reflected between the object holding plate and the object due to the physical properties of the object holding plate and the object described later, and will not propagate to the acoustic detector. Thus, the crossing angle φ1-02 of the acoustic detectors was set to 60 degrees. An acoustic wave propagation medium is placed between the acoustic detector and the object holding plate. The sound source is a circle having a diameter of 1 mm, and was placed at a location that is 10 mm apart from the interface of the object holding plate and the object at an equal distance from both acoustic detectors. The propagation velocity of the sound wave was 1500 (m/s) in the acoustic wave propagation medium, 2200 (m/s) in the object holding plate, and 1500 (m/s) in the object. The density was 1 (g/cm³) in the acoustic wave propagation medium, 0.83 (g/cm³) in the object holding plate, and 1 (g/cm³) in the object.

Simulation was performed based on the foregoing system, and the obtained sound pressure distribution is shown in FIG. 13B. As with the comparative example, an image caused by acoustic interference appears in the acoustic wave propagation medium, but in reality attention is given only to the object, and only the inside of the object is obtained as the result. The dark portion shown at the center of the object is the sound source that is obtained based on the back projection.

Both sound sources are a circle having a diameter of 1 mm and, upon comparing the lateral size of the image of the sound source, while the planar type was approximately 2 mm, it was confirmed that the lateral resolution improved in the present invention whereby the size was approximately 1 mm.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-086569, filed Apr. 8, 2011, which is hereby incorporated by reference herein in its entirety.

1. A measuring apparatus, comprising:
a holding unit holding an object;
an acoustic detecting unit including at least one detector which receives, via said holding unit, an acoustic wave that is generated from the object which is being irradiated with light, and converts the acoustic wave into an electrical signal; and
a processor generating image data of the object by using the electrical signal based on the acoustic wave that has been received by said acoustic detecting unit at a first measurement location and a second measurement location,
wherein said acoustic detecting unit is arranged so as to form an overlapped area in which an effective receiving area of said detector in the first measurement location and an effective receiving area of said detector in the second measurement location overlap in the object.

2. The measuring apparatus according to claim 1, wherein said acoustic detecting unit includes a first detector that is arranged in the first measurement location and a second detector that is arranged in the second measurement location.

3. The measuring apparatus according to claim 1, wherein
said acoustic detecting unit includes one detector, and
said detector receives the acoustic wave in the first measurement location and the second measurement location.

4. The measuring apparatus according to claim 1, wherein the first measurement location and the second measurement location are arranged on the same side, via the holding unit, relative to the object.

5. The measuring apparatus according to claim 4, wherein a central axis of the effective receiving area of said detector in the first measurement location and a central axis of the effective receiving area of said detector in the second measurement location are line-symmetric relative to a normal direction of an interface between said holding unit and the object.

6. The measuring apparatus according to claim 1, wherein an angle θ1 between a detection face of said detector and said holding unit, the directional angle θ2 of said detector, and a critical angle θ3 of an acoustic wave which is generated inside of the object at a boundary between the object and said holding unit, satisfy the following expression;
0<θ1≦3−θ2.

7. The measuring apparatus according to claim 1, wherein
said holding unit includes two members which hold the object from either side, and
the first measurement location and the second measurement location are respectively arranged on said two members.

8. The measuring apparatus according to claim 1, further comprising:
a scanning controller which causes said acoustic detecting unit to perform scanning.

9. The measuring apparatus according to claim 1, wherein said processor generates image data of the object by using both an electrical signal converted from an acoustic wave that has been detected at the first measurement location and an electrical signal converted from an acoustic wave that has been detected at the second measurement location.

10. The measuring apparatus according to claim 1, wherein said processor generates first image data using an electrical signal converted from an acoustic wave that has been detected at the first measurement location, generates second image data using an electrical signal converted from an acoustic wave that has been detected at the second measurement location, and generates image data of the object by using the first image data and the second image data.

11. The measuring apparatus according to claim 1, further comprising:
a scanning controller which causes said acoustic detecting unit to perform scanning, wherein
said acoustic detecting unit includes a first detector that is arranged at the first measurement location and a second detector that is arranged at the second measurement location, and
said scanning controller causes said first detector and said second detector to perform scanning without changing the relative placement of said first detector and said second detector.

12. The measuring apparatus according to claim 1, wherein said acoustic detecting unit is arranged so that the overlapped area becomes an overlapped area that is thicker than the object in a normal direction of an interface between said holding unit and the object.
